(19) United States
(12) Patent Application Publication
(10) Pub. No.: US 2020/0303058 A1
(43) Pub. Date: Sep. 24, 2020

(54) LIFESTYLE HABIT MANAGEMENT DEVICE, METHOD, AND NON-TRANSITORY RECORDING MEDIUM IN WHICH PROGRAM IS STORED

(71) Applicants: OMRON Corporation, Kyoto (JP); OMRON HEALTHCARE Co., Ltd., Kyoto (JP)

(72) Inventors: Toru DENO, Kyoto (JP); Naoki TSUCHIYA, Kyoto (JP); Hiroshi USUI, Kyoto (JP); Kosuke INOUE, Kyoto (JP); Yoshiyuki MORITA, Kyoto (JP); Yasushi MATSUOKA, Kyoto (JP)

(21) Appl. No.: 16/894,993

(22) Filed: Jun. 8, 2020

Related U.S. Application Data
(63) Continuation of application No. PCT/JP2018/043768, filed on Nov. 28, 2018.

(30) Foreign Application Priority Data
Dec. 18, 2017 (JP) 2017-241925

(51) Int. Cl. G16H 20/70; G16H 20/10; G16H 50/30; A61B 5/00; A61B 5/021

(52) U.S. Cl. CPC G16H 20/70; G16H 20/10; A61B 5/02108; A61B 5/7275; A61B 5/681; G16H 50/30

(57) ABSTRACT

A lifestyle habit management device according to one aspect of the present invention includes a biological information acquisition unit configured to acquire a measurement result of biological information about a user, a biological information fluctuation detection unit configured to detect fluctuations in the biological information caused by a lifestyle habit to be managed, and a lifestyle habit information generation unit configured to generate lifestyle habit information indicating a history in which the user has performed the lifestyle habit, based on a detection result of the fluctuations in the biological information.

This is a continuation of International Application PCT/JP2018/043768, with an international filing date of Nov. 28, 2018, and International Application JP 2017-241925 with an international filing date of Dec. 18, 2017, filed by applicant, the disclosure of which is hereby incorporated by reference in its entirety.

### TECHNICAL FIELD

The present invention relates to a lifestyle habit management device, a method, and a non-transitory recording medium in which a program is stored that manage the performance of a lifestyle habit by a user.

### BACKGROUND ART

In recent years, the development of wearable-type biological information measurement devices has been advanced. Using a wearable-type biological information measurement device allows a user to constantly measure his/her biological information while going through daily life. The biological information measured by the wearable-type biological information measurement device can be used in various situations such as health care.

For example, JP 2017-27157 A discloses a proposal device that estimates a health state of a user, based on user information including a measurement result of measured biological information such as blood pressure or a heart rate by a wearable terminal, and proposes an appropriate insurance plan to the user, based on an estimated result. In the proposal device, the user information includes a smoking status indicating the number of cigarettes smoked by the user. The smoking status is managed based on the number of times a cigarette is lighted by a terminal device such as a smart lighter.

In the proposal device disclosed in JP 2017-27157 A, for example, when a cigarette is lit by a general-purpose lighter, smoking by the user is not reflected in the smoking status. Thus, the smoking status acquired by the proposal device may not be accurate.

### SUMMARY OF INVENTION

The present invention has been made in view of the above-described circumstances, and an object thereof is to provide a lifestyle habit management device and a method capable of accurately managing the performance of a lifestyle habit by a user.

In order to solve the problem described above, the present invention adopts the following configuration.

A lifestyle habit management device according to one aspect of the present invention includes a biological information acquisition unit configured to acquire a measurement result of biological information about a user, a biological information fluctuation detection unit configured to detect fluctuations in the biological information caused by a lifestyle habit to be managed, and a lifestyle habit information generation unit configured to generate lifestyle habit information indicating a history in which the user has performed the lifestyle habit, based on a detection result of the fluctuations in the biological information.

According to the configuration described above, it is possible to detect that the user has performed the lifestyle habit to be managed from the measurement result of the biological information about the user. Thus, the user does not need to perform a predetermined operation, such as lighting a cigarette by using a specific terminal device. As a result, the performance of the lifestyle habit by the user can be accurately managed.

In the lifestyle habit management device according to the aspect described above, the biological information fluctuation detection unit may detect the fluctuations in the biological information by performing pattern recognition on a waveform of the biological information based on the measurement result. According to the configuration, the fluctuations in the biological information caused by the lifestyle habit to be managed can be accurately detected. For example, it is possible to determine whether the fluctuations in the biological information are caused by the lifestyle habit to be managed or caused by other factors.

In the lifestyle habit management device according to the aspect described above, the biological information fluctuation detection unit may include a waveform fluctuation specification unit configured to specify a fluctuating portion that satisfies a preset condition from a waveform of the biological information based on the measurement result, a lifestyle habit selection unit configured to select a lifestyle habit that has caused the specified fluctuating portion from a plurality of the lifestyle habits to be managed, and a detection result generation unit configured to generate the detection result including information indicating the selected lifestyle habit.

According to the configuration described above, when there are a plurality of lifestyle habits to be managed, it is possible to determine which lifestyle habit of the lifestyle habits has been performed by the user. As a result, the performance of various lifestyle habits by the user can be managed.

In the lifestyle habit management device according to the aspect described above, the lifestyle habit information generation unit may generate the lifestyle habit information that includes information indicating the number of times the user has performed the lifestyle habit. According to the configuration, a history of performance of the lifestyle habit to be managed, such as the number of cigarettes smoked, can be managed.

In the lifestyle habit management device according to the aspect described above, the lifestyle habit includes at least one of smoking, drinking, or medication, for example. According to the configuration, a history in which the user has performed at least one of smoking, drinking, or medication can be managed.

In the lifestyle habit management device according to the aspect described above, the biological information includes blood pressure, for example. According to the configuration, the lifestyle habit information indicating a history in which the user has performed the lifestyle habit to be managed is generated based on the fluctuations in blood pressure. The lifestyle habit in which blood pressure fluctuates in response to the performance of the lifestyle habit by the user, such as smoking, drinking, and medication, can be managed.

The lifestyle habit management device according to the aspect described above may further include a risk evaluation unit configured to evaluate a risk of causing development of brain and cardiovascular diseases, based on the lifestyle habit information. According to the configuration, without inputting information related to the performance of the lifestyle habit to be managed by the user, the risk of causing the development of the brain and cardiovascular diseases can be evaluated by taking the lifestyle habit information into consideration, in addition to a measurement result of the biological information about the user.

According to the present invention, the lifestyle habit management device, the method, and the non-transitory recording medium in which a program is stored, capable of accurately managing the performance of a lifestyle habit by a user can be provided.

### BRIEF DESCRIPTION OF DRAWINGS

FIG. 1 is a block diagram illustrating a lifestyle habit management system according to an embodiment.

FIG. 2 is a block diagram illustrating a schematic configuration of a lifestyle habit management system according to an embodiment.

FIG. 3 is a block diagram illustrating an example of a hardware configuration of a blood pressure measurement device illustrated in FIG. 2.

FIG. 4 is a block diagram illustrating an example of a hardware configuration of a lifestyle habit management device illustrated in FIG. 2.

FIG. 5 is a block diagram illustrating an example of a software configuration of the blood pressure measurement device illustrated in FIG. 2.

FIG. 6 is a block diagram illustrating an example of a software configuration of the lifestyle habit management device illustrated in FIG. 2.

FIG. 7 is a block diagram illustrating a configuration example of a fluctuation detection unit illustrated in FIG. 6.

FIG. 8 is a graph schematically illustrating an effect of smoking on blood pressure.

FIG. 9 is a flowchart illustrating an example of a processing procedure of the blood pressure measurement device according to an embodiment.

FIG. 10 is a flowchart illustrating an example of a processing procedure of the lifestyle habit management device according to an embodiment.

### DESCRIPTION OF EMBODIMENTS

Embodiments of the present invention will be described below with reference to the drawings.

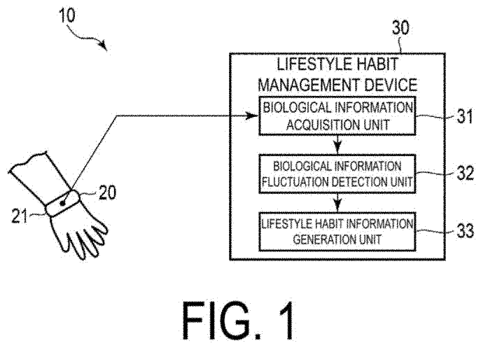

FIG. 1 illustrates a lifestyle habit management system 10 according to an embodiment of the present invention. As illustrated in FIG. 1, the lifestyle habit management system 10 includes a biological information measurement device 20 and a lifestyle habit management device 30. In this example, the lifestyle habit management device 30 is a separate device from the biological information measurement device 20. Note that the lifestyle habit management device 30 may be provided in the biological information measurement device 20.

The biological information measurement device 20 is provided on a wearable device 21 attached to a user being a subject and measures biological information about the user. In the example illustrated in FIG. 1, the wearable device 21 is a wrist-mounted device, and the biological information measurement device 20 performs the measurement of biological information on a wrist as a target measurement site. Note that the target measurement site is not limited to a wrist and may be another site such as an upper arm. The biological information refers to information that can be obtained from a user's body. Examples of the biological information include blood pressure, pulse, a heartbeat, arterial oxygen saturation, blood alcohol concentration, and the like. The biological information measurement device 20 may measure one type of the biological information or may measure a plurality of types of the biological information.

The biological information measurement device 20 transmits measurement data including a measurement result of measured the biological information to the lifestyle habit management device 30. Communication between the biological information measurement device 20 and the lifestyle habit management device 30 is performed by wired communication, wireless communication, or a combination of wired and wireless communications. Note that the measurement data may be provided to the lifestyle habit management device 30 by using a removable medium such as a memory card.

The lifestyle habit management device 30 manages the performance of a lifestyle habit to be managed by the user, based on the measurement result received from the biological information measurement device 20. The lifestyle habit refers to a habit in daily life, which is represented by diet, exercise, recreation, smoking, drinking, medication, and the like. As an example, the lifestyle habit that is closely involved in the development of brain and cardiovascular diseases, such as smoking, drinking, and medication, is to be managed. The lifestyle habit to be managed may be one type of lifestyle habits or a plurality of types of lifestyle habits. In the following, the lifestyle habit to be managed may be referred to as a target lifestyle habit.

The lifestyle habit management device 30 includes a biological information acquisition unit 31, a biological information fluctuation detection unit 32, and a lifestyle habit information generation unit 33.

The biological information acquisition unit 31 acquires a measurement result of measured biological information about a user by the biological information measurement device 20.

The biological information fluctuation detection unit 32 receives the measurement result from the biological information acquisition unit 31 and detects fluctuations in the biological information caused by a target lifestyle habit. In other words, the biological information fluctuation detection unit 32 specifies the target lifestyle habit, based on fluctuations in the biological information acquired from the measurement result. For example, the biological information fluctuation detection unit 32 detects fluctuations in the biological information caused by the target lifestyle habit by performing pattern recognition on a waveform of the biological information based on the measurement result.

The lifestyle habit information generation unit 33 generates lifestyle habit information indicating a history in which the user has performed the target lifestyle habit, based on a detection result of fluctuations in the biological information. The lifestyle habit information includes, for example, information indicating the number of times the user has performed the target lifestyle habit, information indicating whether or not the user has performed the target lifestyle habit, information indicating a length of time the user has performed the target lifestyle habit, or the like. As an example, the lifestyle habit information includes information indicating the number of cigarettes smoked on a daily basis.

The lifestyle habit management device 30 having the configuration described above can detect that the user has performed the target lifestyle habit from the biological information about the user being measured by the biological information measurement device 20 and generates the lifestyle habit information, based on the detection result. The lifestyle habit information is generated without depending on an input from the user, such as an operation of a specific terminal by the user. Thus, the performance of the lifestyle habit by the user can be accurately managed.

The lifestyle habit information generated in this manner can be used in various situations. For example, the lifestyle habit management device 30 may present the lifestyle habit information to the user in order to promote improvement in the lifestyle habit. Further, the lifestyle habit management device 30 may also provide the lifestyle habit information to a third party, such as an insurance company. Insurance companies can use the provided lifestyle habit information for computing an insurance premium. For example, when the lifestyle habit information indicates that a user who is a smoker has successfully quit smoking after taking out insurance, an insurance premium is conceivably reduced when the insurance contract is updated. The converse is also possible.

A specific example of the lifestyle habit management system according to the present embodiment will be described below.

### System Configuration

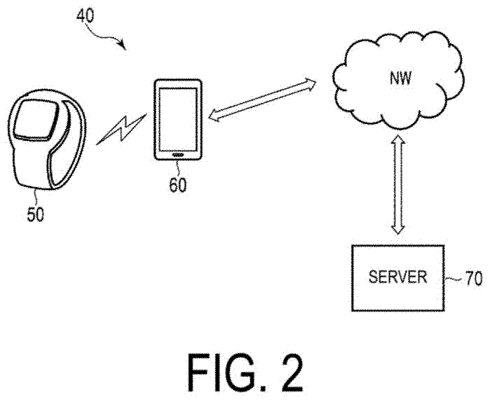

FIG. 2 illustrates a schematic configuration of a lifestyle habit management system 40 according to an example of the present embodiment. As illustrated in FIG. 2, the lifestyle habit management system 40 includes a wristwatch-type blood pressure measurement device 50, a lifestyle habit management device 60, and a server 70.

The blood pressure measurement device 50 corresponds to the biological information measurement device 20 illustrated in FIG. 1. The blood pressure measurement device 50 is attached to a wrist of the user. The blood pressure measurement device 50 measures blood pressure of the user and generates a measurement result. The measurement result includes time series data of blood pressure values, such as systolic blood pressure (SBP) or diastolic blood pressure (DBP), which is not limited thereto. For example, the measurement result may include time series data of a pulse wave (for example, a pressure pulse wave or a volume pulse wave).

The lifestyle habit management device 60 corresponds to the lifestyle habit management device 30 illustrated in FIG. 1. In this example, the lifestyle habit management device 60 is mounted on a portable terminal device owned by the user. The portable terminal device is, for example, a smartphone, a mobile phone, a tablet personal computer (PC), a laptop PC, and the like. Note that the lifestyle habit management device 60 may be mounted on a stationary information processing device (computer) such as a desktop PC. The lifestyle habit management device 60 directly communicates with the blood pressure measurement device 50 and further communicates with the server 70 via a network NW, such as the Internet or a mobile network. Note that the lifestyle habit management device 60 may communicate with the blood pressure measurement device 50 via the network NW.

The lifestyle habit management device 60 receives measurement data including the measurement result from the blood pressure measurement device 50. As described below, the lifestyle habit management device 60 generates lifestyle habit information and risk information, based on the measurement result received from the blood pressure measurement device 50. The lifestyle habit management device 60 transmits user information including the lifestyle habit information and the risk information to the server 70 via the network NW. The server 70 collects and manages user information about a plurality of users from a plurality of lifestyle habit management devices including the lifestyle habit management device 60. The server 70 provides the user information to a third party, such as an insurance company, for example.

### Hardware Configuration

### Blood Pressure Measurement Device

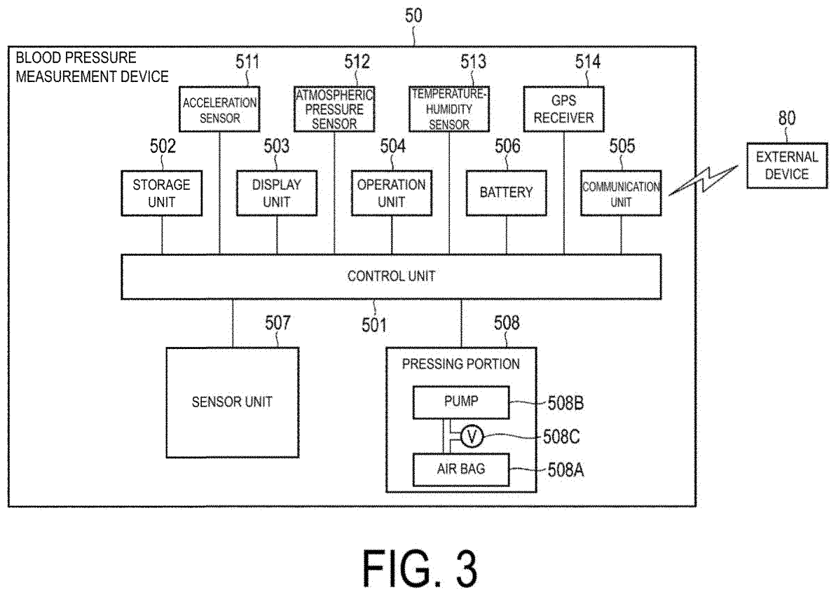

FIG. 3 illustrates an example of a hardware configuration of the blood pressure measurement device 50. The blood pressure measurement device 50 illustrated in FIG. 3 measures a pressure pulse wave by a tonometry method. Here, the tonometry method refers to a method for pressing an artery from above the skin with appropriate pressure, forming a flat portion in the artery, and measuring a pressure pulse wave noninvasively by a pressure sensor in a balanced state between the interior and the exterior of the artery. In the tonometry method, a blood pressure value can be acquired for each heartbeat.

The blood pressure measurement device 50 includes a control unit 501, a storage unit 502, a display unit 503, an operation unit 504, a communication unit 505, a battery 506, a sensor unit 507, and a pressing portion 508.

The control unit 501 includes a central processing unit (CPU), a random access memory (RAM), a read only memory (ROM), and the like and controls each of the components according to information processing. For example, the control unit 501 calculates a blood pressure value, based on an output signal of the sensor unit 507.

The storage unit 502 is an auxiliary storage device such as a semiconductor memory (for example, a flash memory), for example. The storage unit 502 stores a blood pressure measurement program executed by the control unit 501, data about a measurement result including a blood pressure value calculated by the control unit 501, and the like. The blood pressure measurement program is a program for causing the blood pressure measurement device 50 to measure the blood pressure of the user.

The display unit 503 displays information such as a measurement result. For example, a liquid crystal display (LCD), an organic light emitting diode (OLED) display, and the like can be used as the display unit 503. The operation unit 504 allows the user to input an instruction to the blood pressure measurement device 50. The operation unit 504 provides an instruction signal according to an operation by the user to the control unit 501. The operation unit 504 includes a plurality of push buttons, for example. Note that a touch screen may be used as a combination of the display unit 503 and the operation unit 504.

The communication unit 505 is an interface for communicating with an external device 80. The communication unit 505 includes a near-field wireless communication module such as a Bluetooth (trade name) module, which is not limited thereto. The communication unit 505 may include other types of wireless communication modules, such as a Wi-Fi (trade name) module. Further, the communication unit 505 may include a wired communication module. For example, the communication unit 505 may include a micro USB connector and be connected to the external device 80 with a USB cable. The communication unit 505 exchanges data with the external device 80. For example, the communication unit 505 receives measurement data including a measurement result from the control unit 501 and transmits the measurement data to the external device 80. The external device 80 is, for example, the lifestyle habit management device 60 illustrated in FIG. 2.

The battery 506 is, for example, a rechargeable secondary battery. The battery 506 supplies power to each of the components in the blood pressure measurement device 50. The battery 506 supplies power to the control unit 501, the storage unit 502, the display unit 503, the operation unit 504, the communication unit 505, the sensor unit 507, and the pressing portion 508, for example.

The sensor unit 507 is disposed so as to contact a site (a wrist in this example) in which a radial artery is located. The sensor unit 507 includes at least one pressure sensor array on its main surface (surface that contacts the wrist), and the pressure sensor array includes a plurality of (for example, 46) pressure sensors aligned in one direction. An alignment direction of the pressure sensors is a direction that intersects a direction in which the radial artery extends in a state in which the blood pressure measurement device 50 is attached to the user. Each of the pressure sensors detects pressure and generates a pressure signal indicating the detected pressure. As the pressure sensor, a piezoresistive pressure sensor can be used, for example. The pressure signal is amplified by an amplifier and converted to a digital signal by an analog-to-digital converter and is then provided to the control unit 501. A sampling frequency is, for example, 125 Hz.

The pressing portion 508 presses the sensor unit 507 against the wrist. In the tonometry method, a pressure pulse wave and blood pressure are equal under an optimum pressing condition. The pressing portion 508 includes an air bag 508A, a pump 508B that supplies air to the air bag, and an exhaust valve 508C for exhausting air from the air bag. When the pump is driven under control by the control unit 501 so as to increase the internal pressure of the air bag, the sensor unit 507 is pressed against the wrist due to the expansion of the air bag. Note that the pressing portion 508 is not limited to the structure using the air bag and may be achieved by any structure as long as the force of pressing the sensor unit 507 against the wrist can be adjusted.

In the blood pressure measurement device 50, a blood pressure measurement is performed in a state in which the sensor unit 507 is held in an arrangement suitable for measurement by the pressing portion 508. The control unit 501 calculates a blood pressure value, based on a pressure signal output from one pressure sensor selected from among the pressure sensors, for example. The blood pressure value includes SBP and DBP, which is not limited thereto. The control unit 501 stores the calculated blood pressure value in association with additional information including time information in the storage unit 502.

The blood pressure measurement device 50 may further include an acceleration sensor 511, an atmospheric pressure sensor 512, a temperature-humidity sensor 513, and a global positioning system (GPS) receiver 514.

The acceleration sensor 511 is, for example, a three-axis acceleration sensor. The acceleration sensor 511, to the control unit 501, outputs an acceleration signal representing acceleration in three directions orthogonal to one another. The control unit 501 can calculate the amount of activity of the user, based on the acceleration signal. The amount of activity is an index related to the physical activity of the user, such as walking, household chores, and desk work. Examples of the amount of activity include the number of steps, the number of steps by fast walking, the number of steps by going up stairs, a walking distance, consumed calories, and the amount of fat burning. The control unit 501 can also estimate a sleep state of the user by detecting a state of tossing and turning in bed by the user, based on the acceleration signal.

The atmospheric pressure sensor 512 detects atmospheric pressure and outputs atmospheric pressure data to the control unit 501. The atmospheric pressure data can be used for calculating the amount of activity. The number of steps by going up stairs and the like can be more accurately calculated by using the atmospheric pressure data along with the acceleration signal.

The temperature-humidity sensor 513 measures an environmental temperature and humidity around the blood pressure measurement device 50. The temperature-humidity sensor 513 outputs environmental data representing the environmental temperature and the humidity to the control unit 501. The control unit 501 stores the environmental data in association with the time information in the storage unit 502. For example, a temperature (a change in temperature) is considered as one of the factors that may cause fluctuations in human blood pressure. Thus, the environmental data is information that may be a factor of fluctuations in blood pressure of the user.

The GPS receiver 514 receives a GPS signal transmitted from a plurality of GPS satellites and outputs the received GPS signal to the control unit 501. The control unit 501 calculates position information about the blood pressure measurement device 50, i.e., a position of the user wearing the blood pressure measurement device 50, based on the GPS signal.

The above-described additional information associated with a measurement result may include the acceleration signal, the atmospheric pressure data, the environmental data, and the position information.

Note that, in relation to the specific hardware configuration of the blood pressure measurement device 50, a component can be omitted, replaced, and added as appropriate according to an embodiment. For example, the control unit 501 may include a plurality of processors.

### Lifestyle Habit Management Device

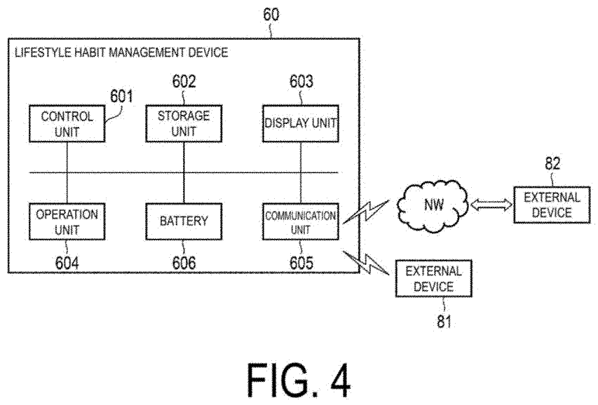

FIG. 4 illustrates an example of a hardware configuration of the lifestyle habit management device 60. As illustrated in FIG. 4, the lifestyle habit management device 60 includes a control unit 601, a storage unit 602, a display unit 603, an operation unit 604, a communication unit 605, and a battery 606.

The control unit 601 includes a CPU, a RAM, a ROM, and the like and controls each of the components according to information processing. The storage unit 602 is an auxiliary storage device such as a hard disk drive (HDD) and a semiconductor memory (for example, a solid-state drive (SSD)), for example. The storage unit 602 stores various data such as a lifestyle habit management program executed by the control unit 601 and measurement data received from the blood pressure measurement device 50. The lifestyle habit management program is a program for causing the lifestyle habit management device 60 to manage performance of a lifestyle habit by the user.

A combination of the display unit 603 and the operation unit 604 is achieved by a touch screen. The touch screen may be either a pressure sensitive type (resistive type) or a proximity type (capacitive type). For example, an LCD, an OLED display, and the like can be used as the display unit 603. The operation unit 604 allows the user to input an instruction to the lifestyle habit management device 60. The operation unit 604 provides an instruction signal according to an operation by the user to the control unit 601. The operation unit 604 may further include a plurality of push buttons. Note that the display unit 603 and the operation unit 604 may be achieved as separate devices. For example, the operation unit 604 may include a keyboard.

The communication unit 605 is an interface for communicating with an external device. In this example, the communication unit 605 includes: a wireless communication module for communicating with an external device 81; and a wireless communication module for communicating with an external device 82. For example, the communication unit 605 includes a Bluetooth module and communicates with the external device 81 in a one-to-one manner. Furthermore, the communication unit 605 includes a Wi-Fi module, is connected to the network NW via a Wi-Fi base station and communicates with the external device 82 via the network NW. Note that the communication unit 605 may include a wired communication module. For example, the communication unit 605 may include a USB connector and be connected to the external device 81 with a USB cable. Note that communication with the external device 81 may follow the same wireless communication standard as that of communication with the external device 82.

The external device 81 is, for example, the blood pressure measurement device 50 illustrated in FIG. 2, and the external device 82 is, for example, the server 70 illustrated in FIG. 2. The communication unit 605 receives measurement data from the blood pressure measurement device 50 and transmits the measurement data to the control unit 601. The communication unit 605 receives user information from the control unit 601 and transmits the user information to the server 70 via the network NW.

The battery 606 is, for example, a rechargeable secondary battery. The battery 606 supplies power to each of the components in the lifestyle habit management device 60. The battery 606 supplies power to the control unit 601, the storage unit 602, the display unit 603, the operation unit 604, and the communication unit 605, for example.

The lifestyle habit management device 60 may further include an acceleration sensor, an atmospheric pressure sensor, a temperature-humidity sensor, and a GPS receiver. These are similar to the acceleration sensor 511, the atmospheric pressure sensor 512, the temperature-humidity sensor 513, and the GPS receiver 514 illustrated in FIG. 3, and thus descriptions thereof will be omitted. Further, the control unit 601 can calculate the amount of activity, positional information, and the like similarly to the description related to the control unit 501 illustrated in FIG. 3.

Note that, in relation to the specific hardware configuration of the lifestyle habit management device 60, a component can be omitted, replaced, and added as appropriate according to an embodiment. For example, the control unit 601 may include a plurality of processors. Further, the lifestyle habit management device 60 may be achieved by a plurality of information processing devices.

### Server

An example of a hardware configuration of the server 70 illustrated in FIG. 2 will be briefly described.

The server 70 is a computer including, for example, a control unit, a storage unit, and a communication unit. The control unit includes a CPU, a RAM, a ROM, and the like and controls each of the components according to information processing. The storage unit is an auxiliary storage device such as an HDD and SSD, for example. The storage unit stores various data such as various programs executed by the control unit and user information received from the lifestyle habit management device 60. The communication unit is an interface for communicating with an external device. The communication unit includes a wired communication module, which is not limited thereto. For example, the communication unit is connected to a router with a local area network (LAN) cable and connected to the network NW via the router and an optical network unit (ONU). The communication unit communicates with an external device (for example, the lifestyle habit management device 60 illustrated in FIG. 2) via the network NW.

### Software Configuration

### Blood Pressure Measurement Device

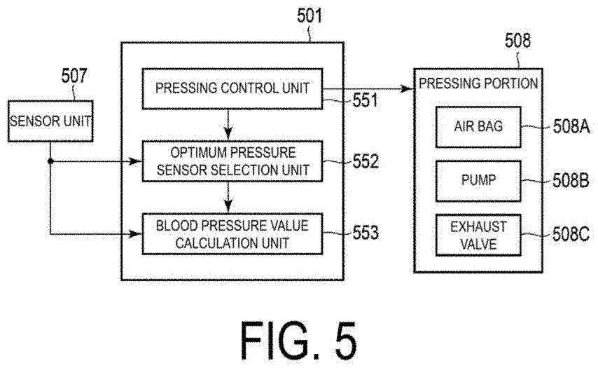

An example of a software configuration of the blood pressure measurement device 50 will be described with reference to FIG. 5.

The control unit 501 (FIG. 3) of the blood pressure measurement device 50 loads the blood pressure measurement program stored in the storage unit 502 into the RAM. Then, the control unit 501 interprets and executes the blood pressure measurement program loaded in the RAM by the CPU and controls each of the components. In this way, as illustrated in FIG. 5, the blood pressure measurement device 50 functions as a computer including a pressing control unit 551, an optimum pressure sensor selection unit 552, and a blood pressure value calculation unit 553.

The pressing control unit 551 controls the pressing portion 508. Specifically, the pressing control unit 551 controls the driving of the pump 508B and the opening and closing of the exhaust valve 508C. The pressing control unit 551 provides a drive signal for driving the pump 508B to the pressing portion 508 in order to supply air to the air bag 508A. The pressing control unit 551 provides a drive signal for opening the exhaust valve 508C to the pressing portion 508 in order to discharge air from the air bag 508A.

The optimum pressure sensor selection unit 552 selects an optimum pressure sensor from among the pressure sensors of the sensor unit 507. When the sensor unit 507 is pressed against the wrist by the pressing portion 508, a flat portion is generated in the radial artery. A pressure pulse wave detected by the pressure sensor located in the flat portion of the radial artery is not affected by the tension of a wall of the radial artery, and an amplitude is the greatest. Further, the pressure pulse wave has the highest correlation with a blood pressure value. Thus, the optimum pressure sensor selection unit 552 determines a pressure sensor that detects a maximum amplitude of the pressure pulse wave as an optimum pressure sensor. The optimum pressure sensor selection unit 552 provides identification information that identifies the pressure sensor selected as the optimum pressure sensor to the blood pressure value calculation unit 553.

The blood pressure value calculation unit 553 receives the identification information from the optimum pressure sensor selection unit 552 and calculates a blood pressure value based on a pressure signal from the optimum pressure sensor indicated by the identification information. The blood pressure value calculation unit 553 extracts a waveform of the pressure pulse wave of one heartbeat, calculates SBP based on a maximum value in the extracted waveform of the pressure pulse wave, and calculates DBP based on a minimum value in the extracted waveform of the pressure pulse wave.

In the present embodiment, an example in which all functions of the blood pressure measurement device 50 are achieved by a general-purpose CPU is described. However, some or all of the functions described above may be achieved by one or a plurality of dedicated processors.

### Lifestyle Habit Management Device

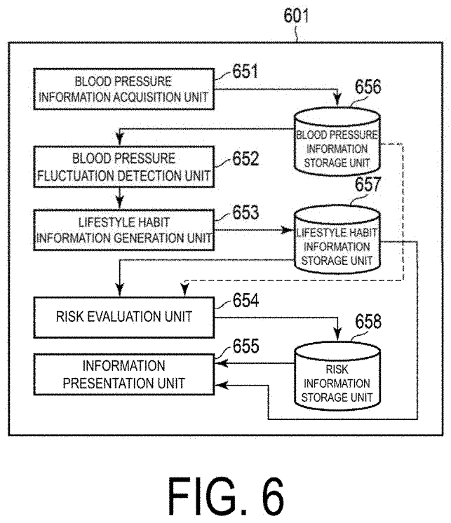

An example of a software configuration of the lifestyle habit management device 60 will be described with reference to FIG. 6.

The control unit 601 (FIG. 4) of the lifestyle habit management device 60 loads the lifestyle habit management program stored in the storage unit 602 into the RAM. Then, the control unit 601 interprets and executes the lifestyle habit management program loaded in the RAM by the CPU and controls each of the components. In this way, as illustrated in FIG. 6, the lifestyle habit management device 60 functions as a computer including a blood pressure information acquisition unit 651, a blood pressure fluctuation detection unit 652, a lifestyle habit information generation unit 653, a risk evaluation unit 654, an information presentation unit 655, a blood pressure information storage unit 656, a lifestyle habit information storage unit 657, and a risk information storage unit 658. The blood pressure information acquisition unit 651, the blood pressure fluctuation detection unit 652, and the lifestyle habit information generation unit 653 respectively correspond to the biological information acquisition unit 31, the biological information fluctuation detection unit 32, and the lifestyle habit information generation unit 33 illustrated in FIG. 1. The blood pressure information storage unit 656, the lifestyle habit information storage unit 657, and the risk information storage unit 658 are achieved by the storage unit 602.

The blood pressure information acquisition unit 651 acquires a measurement result of measured blood pressure of the user by the blood pressure measurement device 50 and stores the acquired measurement result in the blood pressure information storage unit 656. For example, the blood pressure information acquisition unit 651 acquires the measurement result from the blood pressure measurement device 50 via the communication unit 605. As described above, the blood pressure measurement device 50 measures a pressure pulse wave by the tonometry method, and the measurement result includes information indicating a blood pressure value for each heartbeat.

The blood pressure fluctuation detection unit 652 reads the measurement result from the blood pressure information storage unit 656 and detects fluctuations in blood pressure caused by a target lifestyle habit. In the following, the fluctuations in blood pressure caused by the target lifestyle habit may be referred to as observed fluctuations in blood pressure. As an example, the blood pressure fluctuation detection unit 652 detects the observed fluctuations in blood pressure by performing the pattern recognition on a blood pressure waveform generated based on the measurement result. The blood pressure waveform corresponds to time series data of SBP or DBP, for example. In an example in which the measurement result includes a measurement result of a pressure pulse wave, the blood pressure waveform corresponds to an envelope of a waveform of the pressure pulse wave. The blood pressure fluctuation detection unit 652 provides a detection result of the observed fluctuations in blood pressure to the lifestyle habit information generation unit 653. The detection result includes, for example, time information indicating a start time and an end time of the observed fluctuations in blood pressure, which is not limited thereto. The information included in the detection result can be changed according to a method for managing a target lifestyle habit. For example, the detection result may include information indicating a peak value of the observed fluctuations in blood pressure, information indicating an amplitude of the observed fluctuations in blood pressure (for example, a difference between a peak value of the observed fluctuations in blood pressure and a blood pressure value immediately before the occurrence of the observed fluctuations in blood pressure), and the like.

The lifestyle habit information generation unit 653 generates lifestyle habit information indicating a history in which the user has performed the target lifestyle habit, based on the detection result of the observed fluctuations in blood pressure, and stores the generated lifestyle habit information in the lifestyle habit information storage unit 657. For example, the lifestyle habit information generation unit 653 generates the lifestyle habit information that includes the number of times the user has performed the target lifestyle habit. The lifestyle habit information may include information indicating the presence or absence of performance of the target lifestyle habit. As an example, the lifestyle habit information includes information indicating the number of cigarettes smoked, the presence or absence of drinking, and the number of times of medication taken on a daily basis. Whether or not the number of times of smoking a cigarette is reduced and whether or not predetermined medication management is performed can be evaluated by referring to the lifestyle habit information.

The risk evaluation unit 654 reads the lifestyle habit information from the lifestyle habit information storage unit 657. The risk evaluation unit 654 evaluates a risk of causing the development of brain and cardiovascular diseases, based on the read lifestyle habit information, and stores risk information indicating the risk in the risk information storage unit 658. The risk is expressed by sections (levels), for example. As a simple example, the risk evaluation unit 654 evaluates that a risk is “low” when the average number of cigarettes smoked per day is zero, a risk is “medium” when the average number of cigarettes smoked per day is one to nine, and a risk is “high” when the average number of cigarettes smoked per day is 10 or more. Note that the risk may be expressed numerically. The risk evaluation unit 654 may evaluate the risk, based on the lifestyle habit information related to a plurality of types of target lifestyle habits. In addition, the risk evaluation unit 654 may further evaluate the risk, based on the measurement result.

The information presentation unit 655 reads the lifestyle habit information from the lifestyle habit information storage unit 657 and presents the lifestyle habit information to the user. Specifically, the information presentation unit 655 causes the lifestyle habit information to be displayed on the display unit 603. Note that the presentation method is not limited to the display, and other methods such as printing may be used. Further, the information presentation unit 655 reads the risk information from the risk information storage unit 658 and presents the risk information to the user.

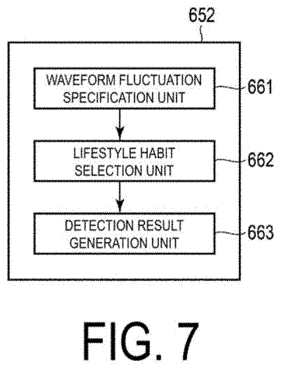

An example of the blood pressure fluctuation detection unit 652 will be described with reference to FIG. 7.

FIG. 7 illustrates an example of the blood pressure fluctuation detection unit 652. As illustrated in FIG. 7, the blood pressure fluctuation detection unit 652 includes a waveform fluctuation specification unit 661, a lifestyle habit selection unit 662, and a detection result generation unit 663.

The waveform fluctuation specification unit 661 specifies, from a blood pressure waveform based on the measurement result, a fluctuating portion that satisfies a preset determination condition. The blood pressure waveform is subjected to pre-processing including smoothing. The above-described determination condition includes, for example, the amount of change in a blood pressure value per predetermined period of time. When there are a plurality of types of target lifestyle habits, the determination condition can be set for each of the target lifestyle habits. Note that the determination condition may also be set in consideration of body information representing physical characteristics of the user, such as age, gender, height, and weight. Specifically, the determination conditions may also be set for each attribute group created based on the body information. For example, a determination condition for a male and a determination condition for a female are set. In this case, the waveform fluctuation specification unit 661 uses the determination condition for the attribute group to which the user belongs. The waveform fluctuation specification unit 661 provides the specified fluctuating portion of the blood pressure waveform to the lifestyle habit selection unit 662.

Note that, when acceleration information is included in the measurement data, it is possible to detect that the user has done exercise from the acceleration information. In this case, the waveform fluctuation specification unit 661 may exclude a blood pressure waveform during a period of the exercise from processing targets. In this way, throughput by the control unit 501 can be reduced.

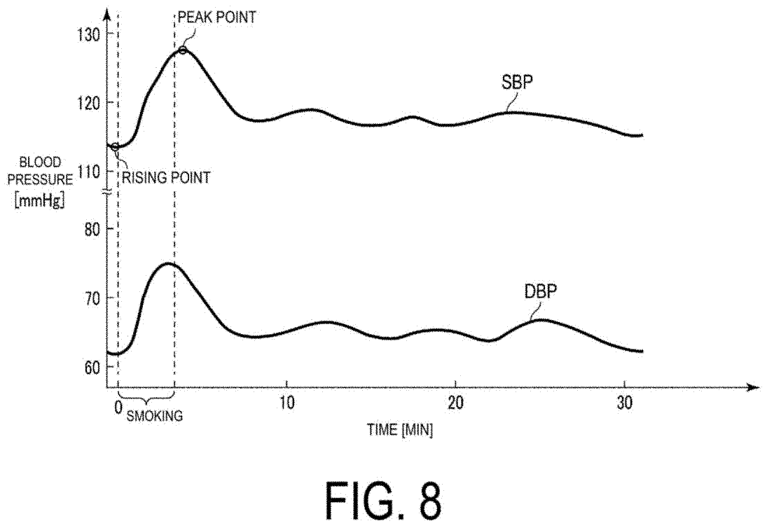

An example of setting a determination condition when the target lifestyle habit is smoking will be described with reference to FIG. 8. FIG. 8 schematically illustrates an effect of smoking on blood pressure. In FIG. 8, a horizontal axis is time, and a vertical axis is blood pressure. As illustrated in FIG. 8, generally, a blood pressure value rapidly rises immediately after a start of smoking, reaches the maximum after two to four minutes since the start of smoking, and gradually descends after an end of smoking. The blood pressure value returns to near a reference value (i.e., the blood pressure value immediately before smoking) after about five minutes since the end of smoking but indicates a value slightly higher than the reference value and requires approximately 30 minutes to return to the reference value. A pulse rate also behaves similarly to the blood pressure value. The determination condition includes, for example, a condition where a peak value (maximum value) of SBP is equal to or greater than, by N [mmHg], a value of SBP at a rising point present before a time at which the peak value is taken and where a difference between the time at the peak value and the time at the rising point is equal to or greater than M [min]. Herein, M and N take specific positive numerical values. Alternatively or additionally, DBP may be used for the determination condition.

When the above-described determination condition is used, the waveform fluctuation specification unit 661 detects a peak point (maximum point) from the pre-processed blood pressure waveform and detects a rising point at a time before the time at the detected peak point. Then, the waveform fluctuation specification unit 661 determines whether or not a difference in a blood pressure value acquired by subtracting the blood pressure value at the rising point from the blood pressure value at the peak point is equal to or greater than a blood pressure threshold value (for example, 10 mmHg) and further determines whether a difference in time acquired by subtracting the time at the rising point from the time at the peak point is equal to or greater than a time threshold value (for example, 1.5 minutes). When the waveform fluctuation specification unit 661 determines that the difference in a blood pressure value is equal to or greater than the blood pressure threshold value and the difference in time is equal to or greater than the time threshold value, the waveform fluctuation specification unit 661 extracts, as a fluctuating portion, a blood pressure waveform in a time range from the time at the rising point to the time after a certain period of time (for example, five minutes) since the time at the peak point.

Further, an example of setting a determination condition when the target lifestyle habit is taking an antihypertensive drug will be briefly described. A way of lowering blood pressure by taking an antihypertensive drug generally varies depending on a type of the antihypertensive drug. Thus, the user inputs the type of the prescribed antihypertensive drug, and a determination condition is set according to the type of the input antihypertensive drug. The determination condition includes, for example, a condition where SBP at a certain point in time is equal to or less than SBP before S minutes from the point in time by T [mmHg]. Herein, S and T take specific positive numerical values. Note that, when a medicine is taken after a meal, the waveform fluctuation specification unit 661 may estimate an end time of the meal from fluctuations in blood pressure and specify a blood pressure waveform in a time section after the end time of the meal as a fluctuating portion.

The lifestyle habit selection unit 662 selects a lifestyle habit that has caused the fluctuating portion of the blood pressure waveform specified by the waveform fluctuation specification unit 661 from a plurality of target lifestyle habits. For example, the pattern recognition is used for the selection. For example, a reference waveform (waveform pattern) corresponding to the target lifestyle habit is prepared in advance, and the lifestyle habit selection unit 662 performs pattern matching, using the reference waveform on the fluctuating portion of the blood pressure waveform. A plurality of reference waveforms may be prepared for each target lifestyle habit. When a degree of similarity between the fluctuating portion of the blood pressure waveform and the reference waveform exceeds a preset similarity degree threshold value, the lifestyle habit selection unit 662 selects a target lifestyle habit corresponding to the reference waveform. When there are a plurality of degrees of similarity that exceed the similarity degree threshold value, the lifestyle habit selection unit 662 selects a target lifestyle habit corresponding to a reference waveform indicating a maximum degree of similarity. When there is no reference waveform in which a degree of similarity with the fluctuating portion of the blood pressure waveform exceeds the preset similarity degree threshold value, the lifestyle habit selection unit 662 determines that the fluctuating portion of the blood pressure waveform specified by the waveform fluctuation specification unit 661 is caused by noise or other factors and rules out the fluctuating portion.

For example, a blood pressure waveform during smoking acquired by a measurement for multiple subjects is collected, the blood pressure waveforms are classified into a plurality of groups, and a representative blood pressure waveform is generated by averaging the blood pressure waveforms belonging to the group for each group. The representative waveform for each group is used as a reference waveform. The group is created based on, for example, the rise amount of blood pressure between the rising point and the peak point. Specifically, a group in which a rise amount of 10 to 12 mmHg, a group in which a rise amount of 12 to 14 mmHg, a group in which a rise amount of 14 to 16 mmHg, and the like are created. Further, the collected blood pressure waveform itself may be used as the reference waveform. The reference waveform can also be generated similarly to other target lifestyle habits such as medication.

Further, the reference waveform may be prepared for each of the attribute groups described above. In this case, the lifestyle habit selection unit 662 uses the reference waveform of the attribute group to which the user belongs.

Note that a learning machine such as a support vector machine (SVM) and a neural network may be used for the pattern recognition. The learning machine is created so as to determine a target lifestyle habit corresponding to the fluctuating portion of the blood pressure waveform specified by the waveform fluctuation specification unit 661 when the fluctuating portion is input. For example, a blood pressure waveform during smoking acquired by a measurement for multiple subjects is collected as learning data, and the learning machine learns by using the learning data.

The detection result generation unit 663 generates a detection result including information indicating the target lifestyle habit selected by the lifestyle habit selection unit 662. For example, the detection result includes identification information, a start time, an end time, a factor (type of the target lifestyle habit), and the like for fluctuations in blood pressure.

Note that the blood pressure fluctuation detection unit 652 may detect observed fluctuations in blood pressure by a method different from the above-described method using the pattern recognition. For example, the blood pressure fluctuation detection unit 652 may calculate a waveform feature amount for the fluctuating portion of the blood pressure waveform specified by the waveform fluctuation specification unit 661 and determine whether or not the fluctuating portion of the blood pressure waveform is observed fluctuations in blood pressure, based on the calculated waveform feature amount.

In the present embodiment, an example in which all functions of the lifestyle habit management device 60 are achieved by a general-purpose CPU is described. However, some or all of the functions described above may be achieved by one or a plurality of dedicated processors.

### Operation

### Blood Pressure Measurement Device

An operation example of the blood pressure measurement device 50 according to the present embodiment will be described.

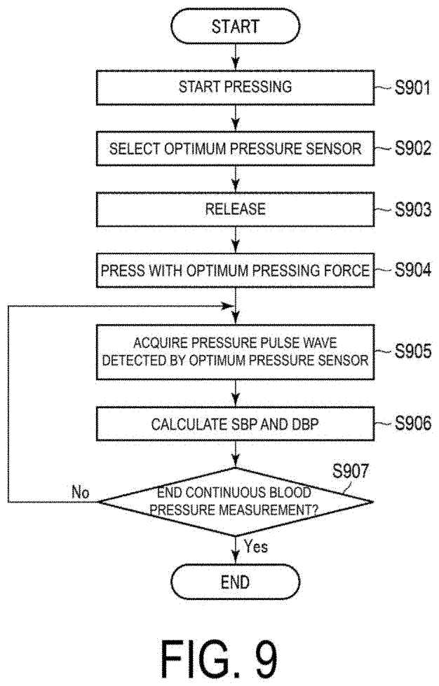

FIG. 9 illustrates an example of an operation in a continuous blood pressure measurement mode of the blood pressure measurement device 50. In step S901, the control unit 501 of the blood pressure measurement device 50 functions as the pressing control unit 551 and drives the pump so as to supply air to the air bag of the pressing portion 508, and thus pressing force on the radial artery by the main surface of the pressing portion 508 gradually increases.

In step S902, the control unit 501 functions as the optimum pressure sensor selection unit 552 and selects the optimum pressure sensor from among the pressure sensors. Specifically, the control unit 501 determines, as the optimum pressure sensor, the pressure sensor that detects a pressure pulse wave at a maximum amplitude in the process of increasing the pressing force. Furthermore, the control unit 501 determines, as optimum internal pressure, the internal pressure of the air bag when the pressure pulse wave at the maximum amplitude is detected.

In step S903, the control unit 501 functions as the optimum pressure sensor selection unit 552, stops the pump, and opens the exhaust valve so as to discharge air in the air bag. In step S904, the control unit 501 closes the exhaust valve, drives the pump such that the internal pressure of the air bag is the optimum internal pressure, and holds a state in which the internal pressure of the air bag is the optimum internal pressure. In this way, a state in which the sensor unit 507 is pressed against the wrist with appropriate pressing force is held.

In step S905, the control unit 501 functions as the blood pressure value calculation unit 553 and acquires the pressure pulse wave detected by the optimum pressure sensor determined in step S902. In step S906, the control unit 501 calculates SBP and DBP from the pressure pulse wave of one heartbeat.

When the control unit 501 does not receive an end instruction for the continuous blood pressure measurement (step S907: No), the control unit 501 returns the processing to step S905. When the control unit 501 receives the end instruction for the continuous blood pressure measurement (step S907: Yes), the control unit 501 ends the processing. In other words, the control unit 501 continues the blood pressure measurement until the control unit 501 receives the end instruction for the continuous blood pressure measurement.

A measurement result acquired in this manner is appropriately provided to the lifestyle habit management device 60. As an example, the control unit 501 periodically attempts the processing of establishing a wireless connection with the lifestyle habit management device 60 and, when the wireless connection is established, controls the control unit 505 so as to transmit measurement data including an untransmitted measurement result to the lifestyle habit management device 60.

Lifestyle Habit Management Device Next, an operation example of the lifestyle habit management device 60 according to the present embodiment will be described.

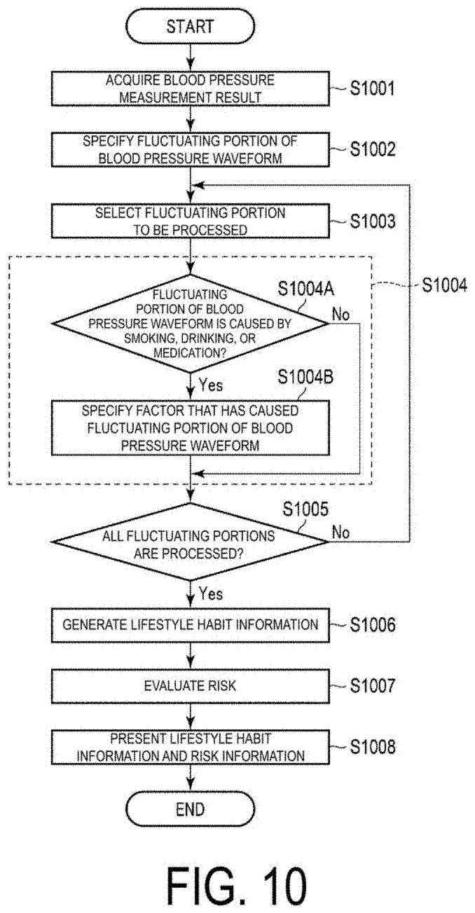

FIG. 10 illustrates an example of a processing procedure of the lifestyle habit management device 60 according to the present embodiment. In this example, a target lifestyle habit is three types of smoking, drinking, and medication.

In step S1001, the control unit 601 of the lifestyle habit management device 60 functions as the blood pressure information acquisition unit 651 and acquires measurement data including a measurement result of measured blood pressure of the user from the blood pressure measurement device 50 via the communication unit 605. In step S1002, the control unit 601 functions as the waveform fluctuation specification unit 661 of the blood pressure fluctuation detection unit 652 and specifies a fluctuating portion of a blood pressure waveform by applying a preset determination condition to the blood pressure waveform.

In step S1003, the control unit 601 selects a fluctuating portion of the blood pressure waveform to be processed from among the fluctuating portions of the blood pressure waveform specified in step S1002.

In step S1004, the control unit 601 functions as the lifestyle habit selection unit 662 of the blood pressure fluctuation detection unit 652 and selects a target lifestyle habit that has caused the fluctuating portion of the blood pressure waveform selected in step S1003. For example, in step S1004A, the control unit 601 determines whether or not the fluctuating portion of the blood pressure waveform is caused by any of smoking, drinking, and medication. Specifically, the control unit 601 performs the pattern matching between the fluctuating portion of the blood pressure waveform and the reference waveform corresponding to each of smoking, drinking, and medication. Note that, when the fluctuating portion of the blood pressure waveform is specified based on, for example, a condition related to smoking in step S1003, it is sufficient that the control unit 601 performs the pattern matching between the fluctuating portion of the blood pressure waveform and the reference waveform corresponding to smoking. When all degrees of similarity between the fluctuating portion of the blood pressure waveform and the reference waveforms are equal to or less than a preset similarity degree threshold value, the control unit 601 determines that the fluctuating portion of the blood pressure waveform is caused by a factor different from smoking, drinking, and medication and proceeds the processing to step S1005. When at least one of the degrees of similarity between the fluctuating portion of the blood pressure waveform and the reference waveforms exceeds the similarity degree threshold value, the control unit 601 proceeds the processing to step S1004B. In step S1004B, the control unit 601 selects a target lifestyle habit corresponding to the reference waveform indicating the highest degree of similarity. In this way, any one of smoking, drinking, and medication is selected as the factor that has caused the fluctuating portion of the blood pressure waveform.

In step S1005, the control unit 601 determines whether or not there is an unprocessed fluctuating portion among the fluctuating portions of the blood pressure waveform specified in step S1002. When there is an unprocessed fluctuating portion, the control unit 601 returns the processing to step S1003. The processing in step S1003 and step S1004 is repeated. When all of the fluctuating portions of the blood pressure waveform specified in step S1002 are processed, the control unit 601 proceeds the processing to step S1006.

In step S1006, the control unit 601 functions as the lifestyle habit information generation unit 653 and generates lifestyle habit information indicating a history in which the user has performed the target lifestyle habit, based on a detection result of the fluctuations in blood pressure. For example, the processing from step S1002 to step S1006 is performed on the measurement result for one day, and thus the lifestyle habit information is updated. For example, the lifestyle habit information includes information indicating the number of cigarettes smoked, the presence or absence of drinking, and whether a medicine is appropriately taken on a daily basis.

In step S1007, the control unit 601 functions as the risk evaluation unit 654, evaluates a risk of causing the development of brain and cardiovascular diseases, based on the lifestyle habit information generated in step S1006, and generates risk information indicating the risk.

In step S1008, the control unit 601 functions as the information presentation unit 655 and presents the lifestyle habit information generated in step S1006 and the risk information generated in step S1007 to the user. In this way, the processing ends.

The control unit 601 may control the communication unit 605 so as to transmit user information including the lifestyle habit information and the risk information to the server 70. The user information is transmitted to the server 70 periodically (for example, weekly), at timing at which the user gives an instruction, or in response to a request from the server 70, for example.

### Effect

As described above, the lifestyle habit management device 60 according to the present embodiment can detect that a user has performed a target lifestyle habit, from blood pressure of the user being continuously measured by the blood pressure measurement device 50, and generates lifestyle habit information, based on the detection result. The lifestyle habit information is generated without depending on a user input, such as an operation on a specific terminal by the user. Thus, the performance of the lifestyle habit by the user can be accurately managed. Since a measurement result is used, the lifestyle habit in which blood pressure fluctuates in response to the performance of the lifestyle habit by the user, such as smoking, drinking, and medication, can be managed.

The pattern recognition may be used in order to detect fluctuations in blood pressure caused by a target lifestyle habit. The fluctuations in blood pressure caused by the target lifestyle habit can be accurately detected by using the pattern recognition. For example, it is possible to determine whether the fluctuations in blood pressure are caused by a target lifestyle habit or caused by other factors. When there are a plurality of target lifestyle habits, a determination condition or a reference waveform is prepared for each of the target lifestyle habits. In this way, it is possible to determine which target lifestyle habit has been performed by the user. As a result, the performance of various lifestyle habits by the user can be managed.

The target lifestyle habit may include at least one of smoking, drinking, or medication, for example. In this way, a history in which the user has performed at least one of smoking, drinking, or medication can be managed. The lifestyle habit information may include information indicating the number of times the user has performed the target lifestyle habit. In this way, a history in which the user has performed the target lifestyle habit, such as the number of cigarettes smoked, can be managed.

The lifestyle habit management device 60 can also evaluate a risk of causing the development of brain and cardiovascular diseases, based on the lifestyle habit information. In this way, without inputting information related to the performance of the target lifestyle habit by the user, the risk of causing the development of the brain and cardiovascular diseases can be evaluated by taking the lifestyle habit information into consideration, in addition to a measurement result of measured biological information about the user.

### Modified Example

In the example described above, the lifestyle habit management device 60 is mounted on a portable terminal device. The lifestyle habit management device 60 may be mounted on other devices, such as the blood pressure measurement device 50 or the server 70, for example. Further, the lifestyle habit management device 60 may also be distributed in a plurality of devices. For example, the blood pressure information acquisition unit 651, the blood pressure fluctuation detection unit 652, the lifestyle habit information generation unit 653, the information presentation unit 655, the blood pressure information storage unit 656, and the lifestyle habit information storage unit 657 may be included in the portable terminal device, and the risk evaluation unit 654 and the risk information storage unit 658 may be included in the server 70.

In the example described above, the blood pressure measurement device 50 adopts the tonometry method. The blood pressure measurement device 50 may be any type of a blood pressure measurement device capable of continuously measuring blood pressure such that blood pressure for each heartbeat can be acquired. For example, a blood pressure measurement device that detects a pulse transit time (PTT) being a transit time of a pulse wave propagating through an artery and that estimates a blood pressure value (for example, SBP and DBP), based on the detected pulse transit time, may be used. Further, a blood pressure measurement device that optically measures the volume pulse wave and calculates a blood pressure value from a measurement result may be used. Further, a blood pressure measurement device that measures blood pressure by using ultrasonic waves may be used.

The blood pressure measurement device 50 may further include a cuff that presses a target measurement site of a user and may have a function of measuring blood pressure according to an oscillometric method, for example. Data about blood pressure measured by using the cuff may be used for calibrating blood pressure acquired by continuous measurements.

The blood pressure measurement device 50 that adopts the tonometry method can measure a pulse simultaneously with blood pressure. Fluctuations in blood pressure caused by a lifestyle habit to be managed can be more accurately detected by also taking fluctuations in pulse rate into consideration, along with the fluctuations in blood pressure.

The present invention is not limited to the above-described embodiment as is, and the components can be modified and embodied within a range that does not depart from the gist of the embodiment in a stage of implementation. Further, various inventions can be formed by an appropriate combination of the plurality of components disclosed in the above-described embodiment. For example, several components may be deleted from all of the components indicated in the embodiment. Furthermore, components in a different embodiment may be appropriately combined.

Some or all of the embodiments described above may also be described as supplementary notes below, which are not limited thereto.

### Supplementary Note 1

A lifestyle habit management device, including:

a processor; and

a memory connected to the processor, wherein

the processor is configured to:

acquire a measurement result of measured biological information about a user,

detect fluctuations in the biological information caused by a lifestyle habit to be managed, and

generate lifestyle habit information indicating a history in which the user has performed the lifestyle habit, based on a detection result of the fluctuations in the biological information.

### Supplementary Note 2D

A lifestyle habit management method, including:

acquiring a measurement result of measured biological information about a user by using at least one processor,

detecting fluctuations in the biological information caused by a lifestyle habit to be managed by using the at least one processor, and

generating lifestyle habit information indicating a history in which the user has performed the lifestyle habit, based on a detection result of the fluctuations in the biological information, by using the at least one processor.

### REFERENCE SIGNS LIST

10 . . . Lifestyle habit management system
20 . . . Biological information measurement device
21 . . . Wearable device
30 . . . Lifestyle habit management device
31 . . . Biological information acquisition unit
32 . . . Biological information fluctuation detection unit
33 . . . Lifestyle habit information generation unit
40 . . . Lifestyle habit management system
70 . . . Server
80, 81, 8 . . . External device
50 . . . Blood pressure measurement device
501 . . . Control unit
502 . . . Storage unit
503 . . . Display unit
504 . . . Operation unit
505 . . . Communication unit
506 . . . Battery
507 . . . Sensor unit
508 . . . Pressing portion
508A . . . Air bag
508B . . . Pump
508C . . . Exhaust valve
511 . . . Acceleration sensor
512 . . . Atmospheric pressure sensor
513 . . . Temperature-humidity sensor
514 . . . GPS receiver
551 . . . Pressing control unit
552 . . . Optimum pressure sensor selection unit
553 . . . Blood pressure value calculation unit
60 . . . Lifestyle habit management device
601 . . . Control unit
602 . . . Storage unit
603 . . . Display unit
604 . . . Operation unit
605 . . . Communication unit
606 . . . Battery
651 . . . Blood pressure information acquisition unit
652 . . . Blood pressure fluctuation detection unit
653 . . . Lifestyle habit information generation unit
654 . . . Risk evaluation unit
655 . . . Information presentation unit
656 . . . Blood pressure information storage unit
657 . . . Lifestyle habit information storage unit
658 . . . Risk information storage unit
661 . . . Waveform fluctuation specification unit
662 . . . Lifestyle habit selection unit
663 . . . Detection result generation unit

1. A lifestyle habit management device, comprising:
a processor; and
a memory, wherein
the processor is configured to acquire a measurement result of SBP or DBP of a user,
the processor is configured to detect fluctuations in the SBP or DBP caused by a lifestyle habit to be managed that includes at least one of drinking and medication from the measurement result, and
the processor is configured to generate lifestyle habit information indicating a history in which the user has performed the lifestyle habit, based on a detection result of the fluctuations in the SBP or DBP.

2. The lifestyle habit management device according to claim 1, wherein
the processor is configured to detect the fluctuations in the SBP or DBP by performing pattern recognition on a waveform of the SBP or DBP based on the measurement result.

3. The lifestyle habit management device according to claim 1, wherein
the processor is configured to specify a fluctuating portion that satisfies a preset condition from a waveform of the SBP or DBP based on the measurement result,
the processor is configured to select a lifestyle habit that has caused the specified fluctuating portion from a plurality of the lifestyle habits to be managed, and
the processor is configured to generate the detection result including information indicating the selected lifestyle habit.

4. The lifestyle habit management device according to claim 1, wherein
the processor is configured to generate the lifestyle habit information that includes information indicating the number of times the user has performed the lifestyle habit.

5. The lifestyle habit management device according to claim 1, wherein
the lifestyle habit includes the at least one of drinking and medication; and smoking.

6. The lifestyle habit management device according to claim 1, wherein
the processor is configured to evaluate a risk of causing development of brain and cardiovascular diseases, based on the lifestyle habit information.

7. A lifestyle habit management method performed by a lifestyle habit management device, the lifestyle habit management method comprising:
a process of acquiring a measurement result of SBP or DBP of a user;
a process of detecting fluctuations in the SBP or DBP caused by a lifestyle habit to be managed that includes at least one of drinking and medication; and
a process of generating lifestyle habit information indicating a history in which the user has performed the lifestyle habit, based on a detection result of the fluctuations in the SBP or DBP.

8. A non-transitory recording medium in which a program is stored for causing the processor included in the lifestyle habit management device described in claim 1 to acquire, detect and generate.

9. The lifestyle habit management device according to claim 2, wherein
the processor is configured to specify a fluctuating portion that satisfies a preset condition from a waveform of the SBP or DBP based on the measurement result,
the processor is configured to select a lifestyle habit that has caused the specified fluctuating portion from a plurality of the lifestyle habits to be managed, and
the processor is configured to generate the detection result including information indicating the selected lifestyle habit.

10. The lifestyle habit management device according to claim 2, wherein
the processor is configured to generate the lifestyle habit information that includes information indicating the number of times the user has performed the lifestyle habit.

11. The lifestyle habit management device according to claim 3, wherein
the processor is configured to generate the lifestyle habit information that includes information indicating the number of times the user has performed the lifestyle habit.

12. The lifestyle habit management device according to claim 2, wherein
the lifestyle habit includes the at least one of drinking and medication; and smoking.

13. The lifestyle habit management device according to claim 3, wherein
the lifestyle habit includes the at least one of drinking and medication; and smoking.

14. The lifestyle habit management device according to claim 4, wherein
the lifestyle habit includes the at least one of drinking and medication; and smoking.

15. The lifestyle habit management device according to claim 2, wherein
the processor is configured to evaluate a risk of causing development of brain and cardiovascular diseases, based on the lifestyle habit information.

16. The lifestyle habit management device according to claim 3, wherein
the processor is configured to evaluate a risk of causing development of brain and cardiovascular diseases, based on the lifestyle habit information.

17. The lifestyle habit management device according to claim 4, wherein
the processor is configured to evaluate a risk of causing development of brain and cardiovascular diseases, based on the lifestyle habit information.

18. The lifestyle habit management device according to claim 5, wherein
the processor is configured to evaluate a risk of causing development of brain and cardiovascular diseases, based on the lifestyle habit information.

19. A non-transitory recording medium in which a program is stored for causing the processor included in the lifestyle habit management device described in claim 2 to acquire, detect and generate.

20. A non-transitory recording medium in which a program is stored for causing the processor included in the lifestyle habit management device described in claim 3 to acquire, detect, generate, specify and select.
